(19) United States
(12) Patent Application Publication
(10) Pub. No.: US 2008/0103544 A1
(43) Pub. Date: May 1, 2008

(54) Method of treating female sexual dysfunction

(76) Inventor: Richard L. Weiner, Dallas, TX (US)

Correspondence Address: Curtis D. Kinghorn;Sirius Strategic, LLC, 6769 W. Shadow Lake Dr., Lino Lakes, MN, 55014, US

(21) Appl. No.: 11/588,653

(22) Filed: Oct. 28, 2006

(51) Int. Cl. A61N 1/36

(52) U.S. Cl. 607/39

(57) ABSTRACT

A method for treating female sexual dysfunction by subcutaneous electrical stimulation of a peripheral nerve innervating at least a portion of the vulva is disclosed. A lead is placed subcutaneously over a peripheral nerve that innervates at least a portion of the vulva and clitoris. The peripheral nerve is electrically stimulated to cause paresthesia. The method encompasses subcutaneous placement of an electrical lead near any peripheral nerve innervating at least a portion of the vulva and subsequent electrical stimulation of the nerve to cause paresthesia. Further, a method for treating intractable pain of the vulva using percutaneous and subcutaneous peripheral nerve electrostimulation techniques is disclosed.

### BACKGROUND OF THE INVENTION

1. Field of the Invention

This invention relates to a method for subcutaneously electrically stimulating peripheral nerves and in a particular embodiment relates to a method for subcutaneously electrically stimulating one or more peripheral nerves to treat female sexual dysfunction.

2. Description of Related Art

Peripheral nerves are nerves in the body other than the nerves of the brain or spinal cord. Peripheral nerves innervate all the organs of the body and connect these organs to the brain either directly or through the spinal cord.

It has been found that electrically stimulating specific peripheral nerves by utilizing the subcutaneous tissues as the electrical conduit (so called “Subcutaneous Electrical Stimulation” or SQS) has proven very helpful in treating patients with injuries that have resulted in the development of chronic intractable female sexual dysfunction, particularly in such patients who have proven unresponsive to conservative female sexual dysfunction management techniques.

SQS is an accepted alternative for those patients who have failed more conservative female sexual dysfunction management therapies. Clinical experience has shown that when applied to appropriate patients by trained practitioners, SQS can reduce female sexual dysfunction, reduce narcotic intake to manage painful female sexual dysfunction and improve the patient's activity levels and their quality of life. SQS has been recognized to have the following desirable characteristics:

The surgical procedure is relatively simple.
SQS is nondestructive. No known permanent surgical or chemical interruption of nerve pathways occurs.
SQS is reversible. If the patient does not benefit, the device can be turned off or removed. There are no known long-lasting medical or surgical side effects.

Patients can be tested for response prior to implant of the complete system.

The inventor of the present invention also discovered and developed a therapy for electrically stimulating peripheral nerves by placing electrodes in the subcutaneous tissue near a desired nerve. Subcutaneous tissue is the tissue beneath the skin or dermis and above the muscle and fascia. This technique is the subject of U.S. Pat. No. 6,505,075, issued Jan. 7, 2003 entitled “Peripheral Nerve Stimulation Method,” the teachings of which are incorporated herein by reference in their entirety.

Female sexual dysfunction is a term broadly used to describe a variety of maladies or conditions. At the 1998 International Consensus Development Conference of Female Sexual Dysfunction, using the Diagnostic and Statistical Manual of Mental Disorders (DSM-IV) and World Health Organization International Classifications of Diseases (ICD-10), attendees divided female sexual dysfunction “FSD” into four disorders briefly defined as:
Desire disorder, a persistent absence of desire for sexual activity.
Arousal disorder, a persistent inability to attain or maintain sufficient sexual excitement.
Orgasm disorder, a persistent difficulty, delay or absence of orgasm after sufficient stimulation.
Female sexual dysfunction disorder, persistent genital female sexual dysfunction associated with sexual intercourse or stimulation.
   The report from the conference was first published in the Journal of Urology (Vol. 163, No. 3) and reprinted in the Journal of Sex & Marital Therapy (Vol. 27, No. 2). It is also believed that intractable pain of the vulva, a malady in its own right, may also contribute to FSD.

Most experts agree that both biological and psychological factors greatly affect whether and to what extent a woman may experience FSD. Biological factors include such things as vaginismus, vulvar dystrophy, herpes simplex virus, episiotomy scars, strictures, rectal disease, levator ani myalgia, interstitial cystitis, postoperative and postradiation changes and bowel disease, hormonal imbalances, infections (e.g., yeast infections), diseases that have potential side effects affecting sexual response (e.g., diabetes, multiple sclerosis), menopause and altered sexual responses to common medications (e.g., psychoactive medications including antipsychotics, barbiturates and certain antidepressants, amphetamines and related anorexic drugs, narcotics, cardiovascular and antihypertensive medications, hormonal preparations including oral contraceptives, antihistamines or even homeopathic remedies). Psychological factors include such things as stress from everyday life including employment worries, financial worries, pressures of juggling work and family, substance use and abuse, abuse, cultural issues, self image issues, intimacy and relationship issues and depression. What complicates this problem even more is that over time biological problems create psychological problems and vice versa.

It appears that a large number of women experience FSD. A report of a study published in the 1999 Journal of the American Medical Association (JAMA) (Vol. 281, No. 6) found that 43 percent of the 1,749 women interviewed by researchers reported experiencing such events as a lack of interest in sex, inability to achieve orgasm and trouble lubricating in the past year.

Help for FSD typically is available in several forms. Counseling is available through both individual or couples therapy. Where FSD may result from side effects to medication, the medication may be changed or its dosage reduced. Hormonal therapies are often used particularly in menopausal and post-menopausal women. However, despite these treatment options, many women still report having problems with FSD. Many patients with FSD do not favorably respond to these medical treatments. Therefore, there is a need for an additional effective treatment of FSD.

### SUMMARY OF THE INVENTION

A method for treating female sexual dysfunction by subcutaneous electrical stimulation (SQS) is disclosed. A lead is placed subcutaneously in the region of peripheral nerves in the vulvar area which includes the dorsal nerve of the clitoris derived from the deep peroneal nerve as the main nerve that innervates the clitoris. The nerve is electrically stimulated to cause paresthesia. A side effect of the paresthesia is that the nerve is stimulated causing a desirable feeling and often a heightened state of arousal. The method of the invention encompasses subcutaneous placement of an electrical lead near any peripheral nerve causing desirable paresthesia in the vulva area and subsequent electrical stimulation of the nerve to cause paresthesia.

It is therefore an object of the invention in one embodiment to provide a method for subcutaneously electrically stimulating nerves of the vulva to create desirable paresthesia.

It is another object of the invention in one embodiment to provide a method for percutaneously placing leads subcutaneously to create desired paresthesia of the vulva.

It is a further object of the invention in one embodiment to provide a method for treating intractable pain of the vulva.

These and other object of the invention will be clear from the following detailed description of the invention.

### BRIEF DESCRIPTION OF THE DRAWINGS

FIG. 1 is a schematic view of the nerves of the vulva.

FIG. 2 is a schematic view of the hardware used to practice the invention of the present invention.

FIG. 3 is a perspective view of a screener device and a screening lead.

FIG. 4 is a top view of a permanent lead.

FIG. 5 is a perspective view of an implantable pulse generator (IPG).

FIG. 6 is a perspective view of an RF system receiver and an RF system transmitter.

FIG. 7 is a top view of an introducer needle curved to facilitate placement of the permanent lead to treat female sexual dysfunction.

FIG. 8 is a schematic view of the entry site used to implant a screening lead or a permanent lead for treating female sexual dysfunction.

FIG. 9 is a close-up schematic view of the placement of the introducer needle prior to placing the screening lead.

FIG. 10 is a schematic view of the placement of the introducer needle with the screening lead being inserted into the introducer needle.

FIG. 11 is a schematic view of the placement of the screening lead, particularly the electrode tip, with the introducer needle removed.

FIG. 12 is a perspective view of a patient prior to being implanted with a permanent lead.

FIG. 13 is a schematic view of the placement of the introducer needle prior to placing the permanent lead.

FIG. 14 is a schematic view of the placement of the introducer needle with the permanent lead being inserted into the introducer needle.

FIG. 15 is a schematic view of the location of the subcutaneous pocket for housing the loop of the permanent lead and the lead anchor.

FIG. 16 is a cutaway view of the location of an alternate embodiment of an electrical stimulator in place.

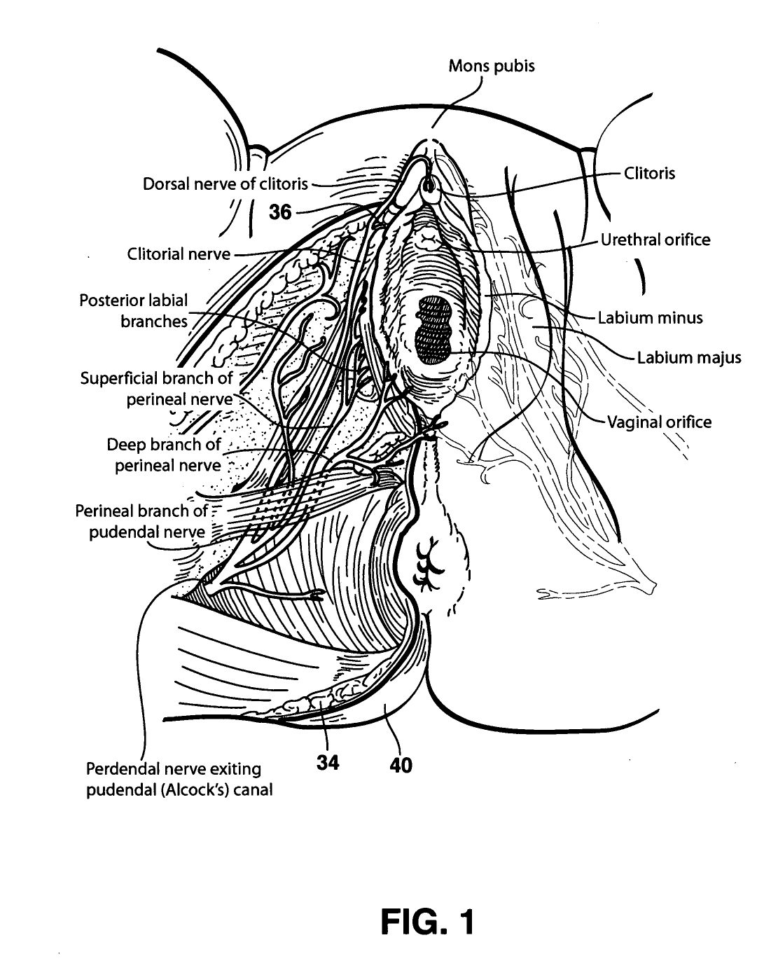

### DETAILED DESCRIPTION OF THE INVENTION

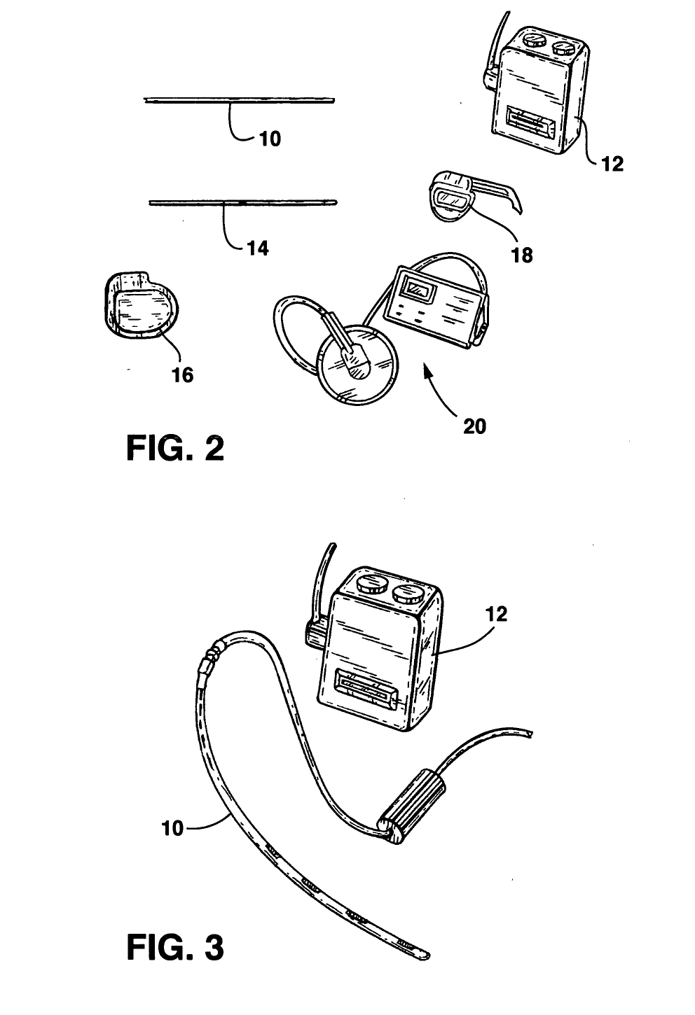

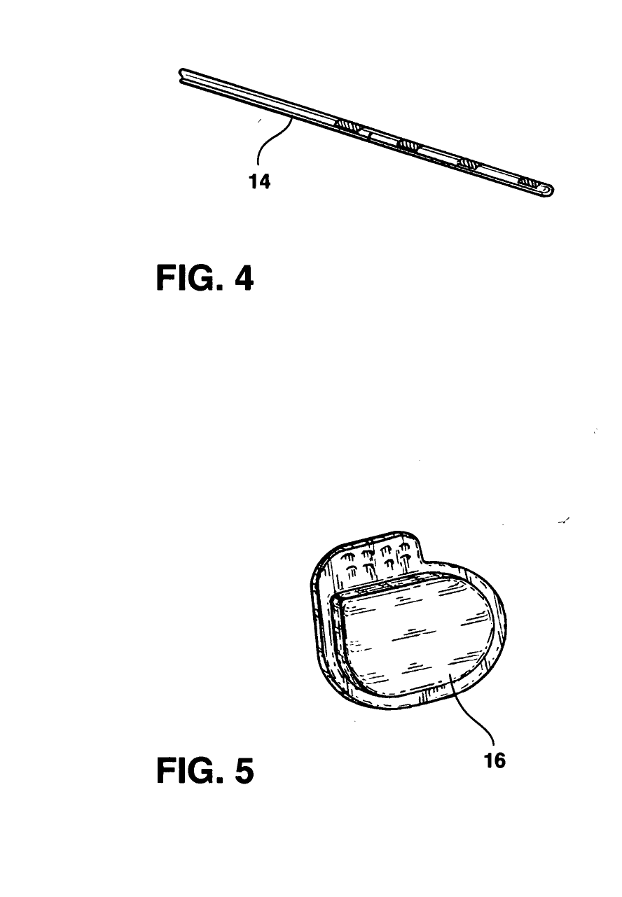

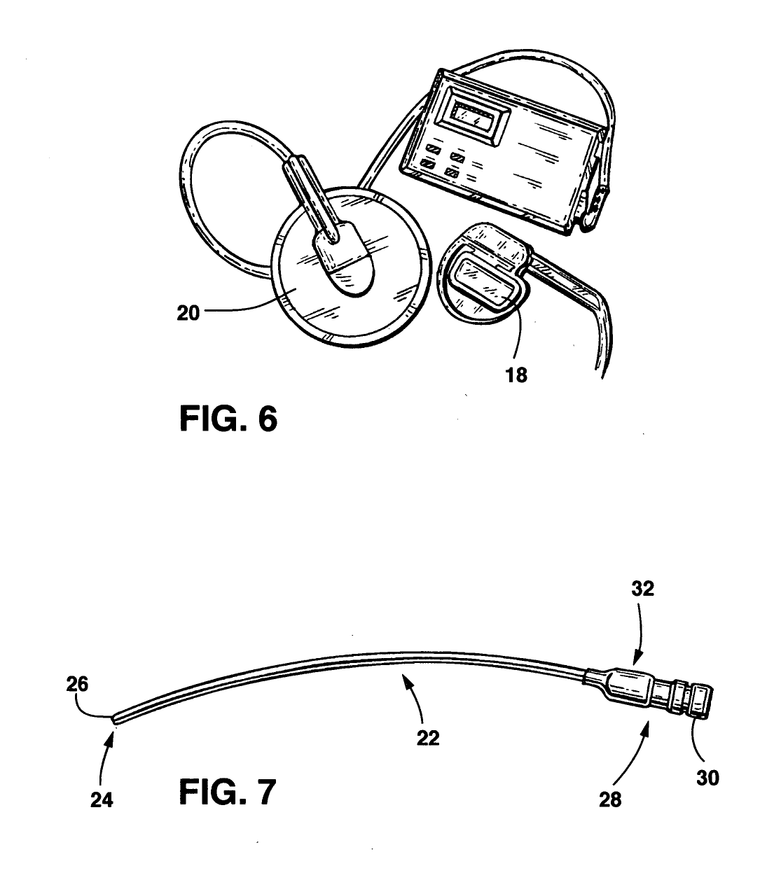

The present invention comprises a method of stimulating peripheral nerves of the vulva and a corresponding method of treating female sexual dysfunction by such stimulation. The method is preferentially accomplished in two stages: a test implantation and screening stage and a permanent implantation of a lead and electrical stimulation system stage. The invention contemplates using, as shown in FIG. 2, a screening lead 10 (shown in detail in FIG. 3), a screener device 12 (also shown in detail in FIG. 3), a permanent lead 14 (shown in detail in FIG. 4) and either an implanted pulse generator (IPG) 16 (shown in detail in FIG. 5) or an implanted RF system receiver 18 and its corresponding RF system transmitter 20 (shown in detail in FIG. 6).

The screening lead 10 and permanent lead 14 are preferably percutaneous leads having the characteristic of being narrow in diameter, very flexible and well tolerated in the subcutaneous space. Commercially available examples of such screening lead 10 or permanent lead 14 are leads from the Pisces-Quad® and TX8™ family of quadripolar and octapolar leads sold by Medtronic, Inc. of Minneapolis, Minn., Linear™ leads sold by Advanced Bionics Corporation of Sylmar, Calif. and Quattrode® and Octrode™ leads sold by Advanced Neuromodulation Systems of Plano Tex. The screening lead 10 is connected to a screener device 12 which provides electrical stimulation pulses to the screening lead 10 to test the placement of and efficacy of the screening lead 10 to treat female sexual dysfunction. Commercially available examples of such screener device 12 are a Model 3625 Screener or a Model 3628 DualScreen® Screener sold by Medtronic, Inc., Precision® trial stimulator device sold by Advanced Bionics Corporation and the model 3510 trial screener sold by Advanced Neuromodulation Systems.

Commercially available examples of IPG 16 are the Synergy® and Restore® devices sold by Medtronic, Inc., the Precision® device sold by Advanced Bionics Corporation and the Genesis® and Eon® devices sold by Advanced Neuromodulation Systems. Commercially available examples of RF system receiver 18 and RF system transmitter 20 are the X-trel® or Matrix® RF Stimulation Systems sold by Medtronic, Inc and the 3416 ans system sold by Advanced Neuromodulation Systems.

The method for treating female sexual dysfunction most preferably involves subcutaneous placement of a lead, most preferably dual permanent leads, on either side of the clitoris within the labia majora which lead or leads stimulate the branches of the pudental and deep peroneal nerves that innervate the skin, clitoris and medial and inferior aspects of the vulva. Sensory fibers from three additional nerves may also be stimulated and include the anterior branch of the ilioninguinal nerve innervating the mons pubis and upper part of the labia majora, the genital femoral nerve innervating the labia majora and the posterior femoral cutaneous nerve supplying the more inferoposterior aspects of the vulva. These peripheral nerves are also preferably subsequently electrically stimulated to cause paresthesia of the vulva area.

Although the method of the present invention preferably contemplates using dual leads to stimulate both sides of the clitoris within the labia majora, a single lead may also be used and located as described. Further, either single, dual or more leads may be placed in the subcutaneous tissue near a particular nerve or group of nerves mentioned to electrically stimulate a particular nerve or group of nerves.

The method also preferably involves placement of a screening lead 10 and subsequent test electrical stimulation prior to placing the permanent lead 14. Although the method preferably involves placing both a screening lead 10 and then a permanent lead 14, the method also includes implanting just the permanent lead 14 as will be described in detail hereafter. For illustration purposes, the method for treating female sexual dysfunction will be described with reference to treating female sexual dysfunction by electrically stimulating the region of the clitoral nerves.

One key to the technical success of this invention is the accurate placement of the permanent lead 14. Because of the importance of accurate placement of the permanent lead 14, accurate placement of permanent lead 14 is facilitated by the placement of the screening lead 10 and the subsequent test electrical stimulation. The steps in the invention to percutaneously place a screening lead 10 to treat female sexual dysfunction will now be described in detail. These steps are given as the preferred method of implementing the invention for most patients. It is recognized, however, that the skilled physician will adapt the method described herein using his or her professional skill and judgment to the particular circumstances of a particular patient.

The first step of the test implantation and screening stage is the implantation of a screening lead 10. The method involves subcutaneous placement of a screening lead 10 in the subcutaneous tissue 34 above (superior to) a nerve innervating the vulva and proximal to such nerve (i.e., between the vulva and the spinal cord). The first step in locating the area to implant the screening lead 10 is to palpate the vulva area to identify the specific nerve that is innervating the area of interest in the vulva. Once the specific nerve innervating the vulva has been identified, an introducer needle 22 is used to place the screening lead 10.

The preferred embodiment for the introducer needle 22 is a Touhy needle. As shown in FIG. 7, the introducer needle 22 has a terminal end 24 that has a beveled edge 26 and a proximal end 28 that includes a hub 30. Beveled edge 26 is a sharp edge that allows the terminal end 24 to be pushed through tissue. Hub 30 allows the physician to manipulate the introducer needle 22. Hub 30 also has a notch 32 that is aligned with the beveled edge 26 to indicate the orientation of beveled edge 26 to the hub 30 by tactile sensation.

The introducer needle 22 is then subcutaneously placed in the subcutaneous tissue 34 above (superior to) the nerve 36 that is innervating the vulva. The subcutaneous tissue 34 is a layer of tissue that lies above (superior to) the fascia 38 but below the dermis 40. Fascia 38 is a sheet of fibrous tissue that envelops the body under the dermis 40 (skin) and also encloses the muscles 42. The dermis 40 is the layer of skin that covers the entire body. As stated, the subcutaneous tissue 34 lies between the fascia 38 and the dermis 40 and is often comprises of fatty tissue. This fatty tissue is highly conductive to electricity. As a result, electrical stimulation of the subcutaneous tissue 34 produces an area of paresthesia that is fairly large compared to the areas of paresthesia created by electrical stimulation of the dermis 40, fascia 38 or muscle 42. This results in effective stimulation of the dysfunctional areas without the need for invasive surgical isolation of specific peripheral nerves utilizing simple percutaneous needle techniques.

In the described method, the introducer needle 22 will be introduced into the fascia 38 so that the introducer needle will be between the patients dermis 40 and fascia 38. The nerves innervating the vulva will be located below the dermis 40 and within the fascia 38. In the case of treating female sexual dysfunction, the introducer needle 22 will be introduced into the subcutaneous tissue 34 so that the introducer needle 22 will lie within the subcutaneous tissues between the patient's dermis 40 and the nerve of interest.

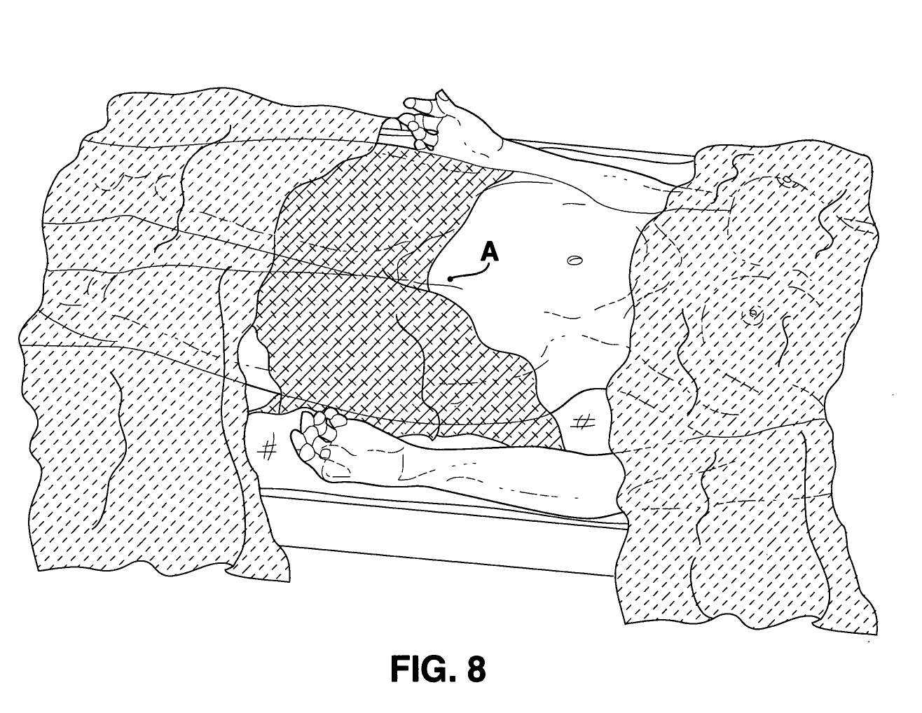

The introducer needle 22 is preferably introduced through a small puncture wound “A” at the needle entry site (FIG. 8). Rapid needle insertion is preferably used. This technique usually obviates the need for even a short acting general anesthetic.

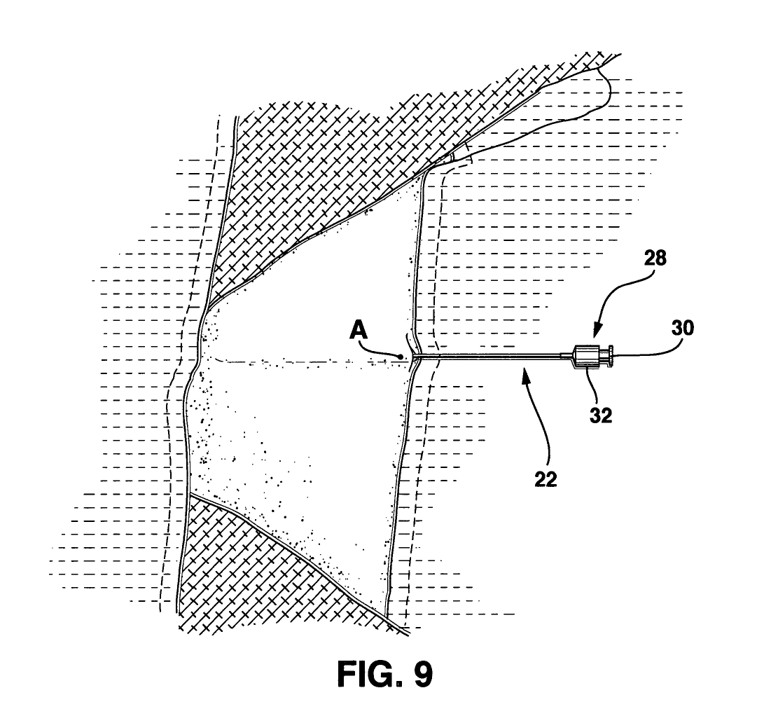

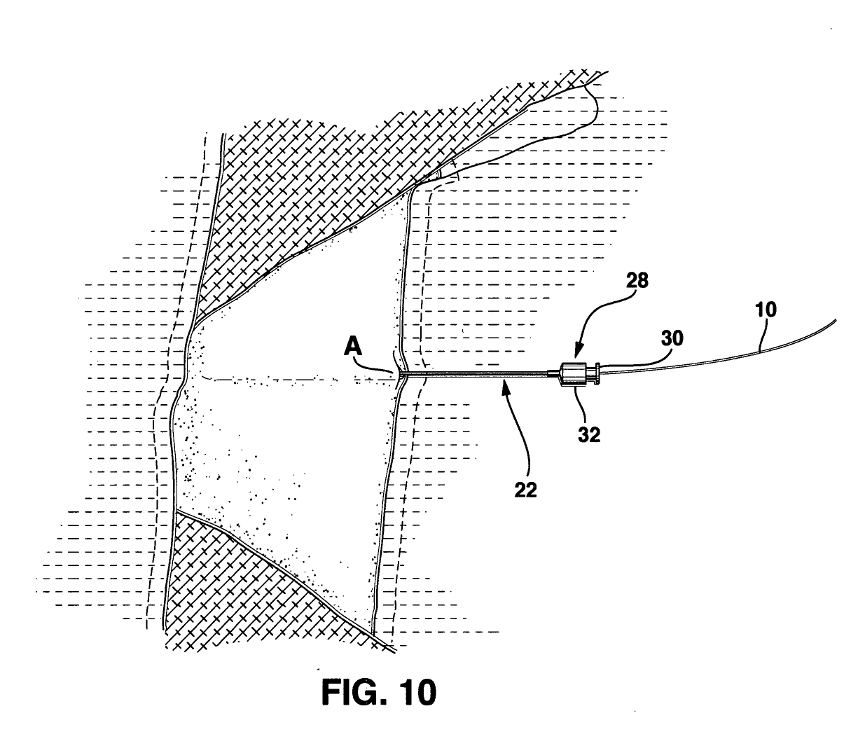

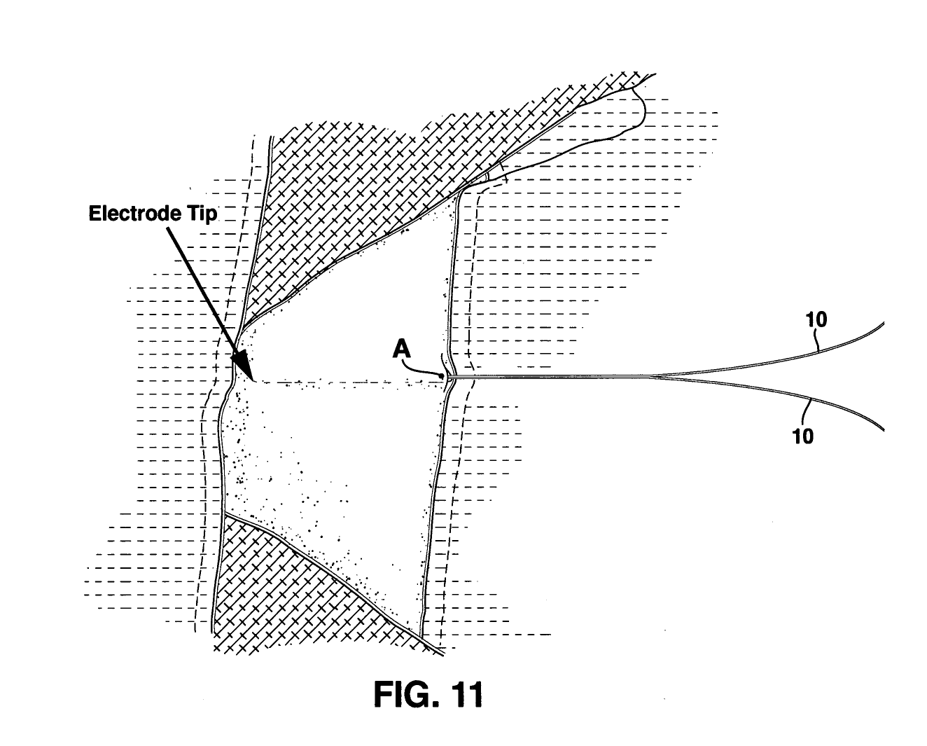

The introducer needle 22 is moved through the subcutaneous tissue 34 to a position over the nerve of interest that innervates the vulva (FIG. 9). When the introducer needle 22 is in position superior to and near the nerve of interest, the screening lead 10 is passed through the introducer needle 22 (FIG. 10) until the screening lead 10 is also in position superior to and near the nerve of interest. Then, the introducer needle 22 is removed leaving the screening lead 10 in place superior to and near the nerve (FIG. 11).

Single or dual quadripolar as well as single or dual octapolar screening leads 10 are preferably used depending on whether it is desirable to stimulate unilaterally (i.e., on one side of the vulva only) or bilaterally (on both sides of the vulva). Where it is desirable to treat the female sexual dysfunction by bilateral stimulation and two screening leads 10 are used (FIG. 11), each screening lead 10 will be placed as described above.

Following placement of the screening lead 10 by the introducer needle 22, the screening lead 10 is connected to the screening device 12, as is well understood in the art. With the screening lead 10 in place as described above and the screening lead 10 connected to the screening device 12, the patient is electrically stimulated by the screening lead 10 and screener device 12 to evaluate the screening lead 10 position and to develop optimal stimulation parameters. Stimulation is applied using the screener device 12 to select various electrode combinations, enabling the patient to report stimulation location, intensity and overall sensation. This allows the physician to test the stimulation and determine optimum stimulation parameters prior to permanently implanting the permanent lead 14 and the source of electrical stimulation pulses, either the IPG 16 or the RF system receiver 18. The effect of this stimulation is determined and the parameters of stimulation adjusted for optimal female sexual dysfunction treatment. It is preferred that the patient be awake and alert so that the patient will provide verbal feedback regarding paresthesia coverage of the vulva area to assist in determining the optimum stimulation parameter settings.

The following have been found to be typical ranges for stimulation parameters for screening by the screener device 12 and the screening lead 10 to optimize paresthesia levels for paresthesia coverage of the vulva. These parameters can vary from patient to patient and may be outside the ranges given here. Never-the-less, these representative values are given for the purpose of illustrating the invention and not for the purpose of limiting the invention. Values for these parameters may be higher or lower than the values shown.

| | | |
|---|---|---|
| Amplitude: | 0.5–4.0 | volts |
| Pulse Width: | 90–300 | microseconds |
| Rate: | 50–400 | Hz |

If the patient reports muscle contractions (grabbing sensation) or burning, this usually indicates that the screening lead 10 is located too close or even too deep to the fascia 34. It may also indicate that the screening lead 10 is not positioned correctly above (superior to) the nerve. It may be necessary to remove and reposition the screening lead 10. If adjustment of screening lead 10 is necessary, the screener device 12 is removed from the screening lead 10. Then, the position of the screening lead 10 is adjusted and stimulation is tested again for optimal paresthesia of the vulva. Adjusting the position of the screening lead 10 may mean removing the screening lead 10 and re-implanting the screening lead 10 according to the technique described above.

After good paresthesia coverage is obtained by manipulating the parameters of stimulation applied through screening lead 10, percutaneous testing wires can be externalized for the test stimulation period as is well understood in the art. This period is used to evaluate the patient's response to stimulation before complete implantation of all system components.

Alternately, once satisfactory paresthesia is confirmed, the screener device 12 may be removed from the screening lead 10 and a source of electrical stimulation pulses such as the IPG 16 or RF system receiver 18 is immediately implanted and attached to the screening lead 10. Hence, screening lead 10 in this embodiment becomes permanent lead 14. However, it is preferred that the patient use the implanted screening lead 10 and screener system 12 for several days prior to implanting a permanent stimulation system.

Once the screening lead 10 has been appropriately positioned and tested, if satisfactory results are obtained, the method should proceed to the “permanent implantation of a lead and electrical stimulation system” stage. The steps in the invention to permanently implant a stimulation system will now be described in detail in connection with the treatment of female sexual dysfunction. As mentioned above, it is possible to implant a source of electrical stimulation pulses such as the IPG 16 or RF system receiver 18 and attached it directly to the screening lead 10 so that screening lead 10 becomes the permanent lead 14. However, the preferred embodiment of the invention contemplates removing the screening lead 10 and replacing it with a permanent lead 14.

After it has been determined that the patient is receptive to the paresthesia from electrically stimulating the peripheral nerve innervating the vulva and the paresthesia associated with the electrical stimulation has been maximized, the screener device 12 is disconnected from the stimulation lead 10 and the screening lead 10 is removed. The patient is then prepared for placement of the permanent lead 14 and the implanted pulse generator (IPG) 16 or implanted RF system receiver 18. The purpose of the “permanent implantation of a lead and electrical stimulation system” stage is to internalize (that is, implant) the permanent lead 14 and either the IPG 16 or the RF system receiver 18. Therefore, this stage includes implanting the permanent lead 14, neurostimulator (either IPG 16 or RF system receiver 18) and any extension sometimes used to connect permanent lead 14 and either IPG 16 or RF system receiver 18 as is well understood in the art.

As stated above, one key to the technical success of this invention is the accurate placement of the permanent lead 14. It is therefore crucial to the success of the invention to have a lead placement for the permanent lead 14 that results in paresthesia that covers the patient's vulva area and particularly the clitoris' 12. Therefore, lead placement is preferably determined using patient feedback during intraoperative testing of the efficacy of the permanent lead 14 placement and the stimulation parameters. Performing implantation of the permanent lead 14 under local anesthetic allows for this feedback.

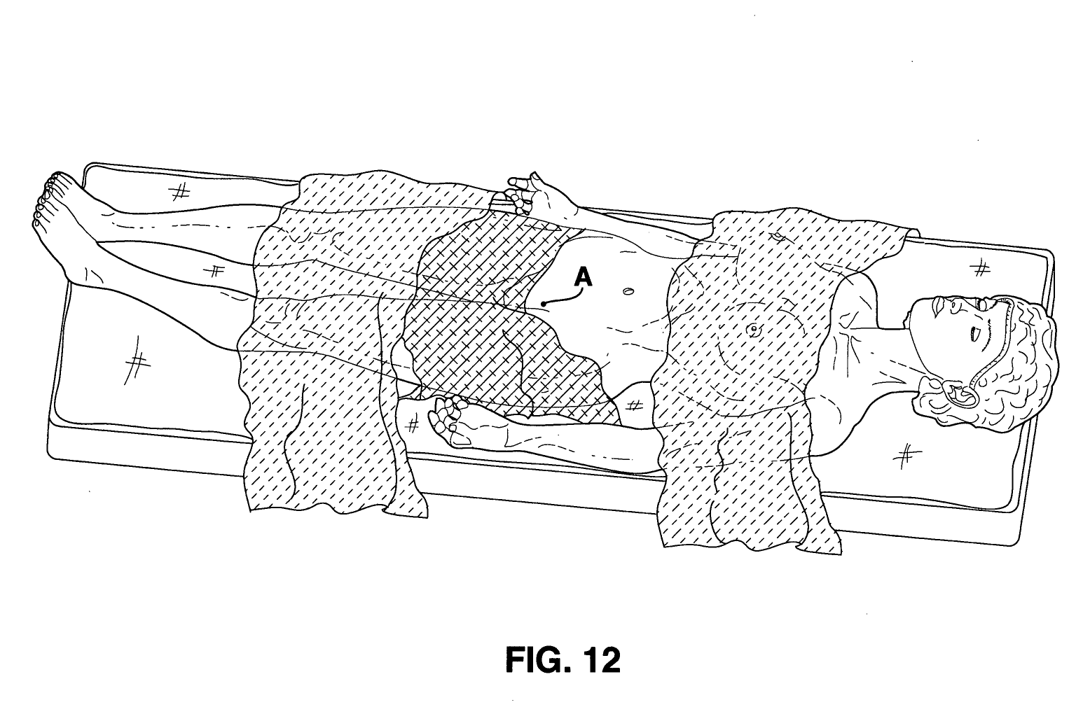

A local anesthetic is preferably used in the area of the introducer needle 22 entry site to ensure the patient is alert and able to respond during the procedure. To help the patient relax, sedatives are also preferably administered intravenously. Prophylactic antibiotics can also be administered intravenously for protection from postoperative infection. As a result, the patient is preferably awake and alert during the placement of the permanent lead 14 and the subsequent test stimulation.

Where treating female sexual dysfunction, the patient is preferably placed in a supine position (lying on the back with the face upward) on the operating room table. (FIG. 12). The patient is prepared and draped according to standard surgical procedure. A Touhy needle is preferably used as an introducer needle 22 to introduce permanent lead 14. The introducer needle 22 includes a stylet 42. The introducer needle 22 is manually gently curved by the physician to conform to the contour of the patient's body superior to and near the targeted peripheral nerve to facilitate placement of the permanent lead 14. Where the targeted peripheral nerves are the pudental and deep peroneal nerves, the introducer needle 22 is manually gently curved by the physician to conform to the suprapubic region to facilitate placement of the permanent lead 14. Where other peripheral nerves are targeted, the introducer needle 22 is manually gently curved by the physician to conform to the region of lead placement to facilitate placement of the permanent lead 14.

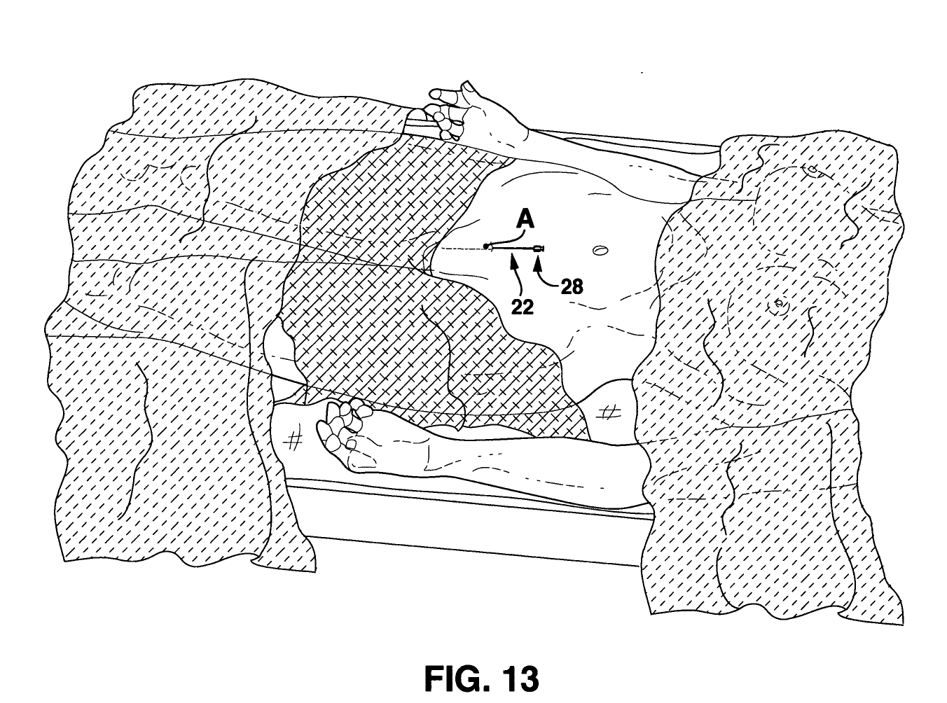

Using local anesthesia, a small puncture wound “A” is made at the needle entry site (FIG. 13) to either side of the midline and superior to the labia majora. The introducer needle 22 is introduced into the subcutaneous tissue 34, superficial to the fascia 38 and muscle 42 but below the dermis 40, without further dissection (cutting so as to separate into pieces or to expose the several parts) across the trunk of the peripheral nerves. These nerves are located within the labia majora/vulva area.

The physician then advances the introducer needle 22 inferiorly from the superior incision point to the appropriate location superior to and near the nerve of interest (FIG. 13). The beveled edge 26 of the introducer needle 22 should face toward the posterior or rear portion of the body. The orientation of the beveled edge 26 can be verified by referring to the notch 32 on the needle hub 30 of the introducer needle 22.

The curve of the introducer needle 22 may be checked, if desired, by the physician removing and re-inserting the needle stylet 42. A useful, curved introducer needle 22 is ensured if it is easy to remove and reinsert the stylet 42 within the introducer needle 22. If desired, an additional check can be made by removing the stylet 42, then carefully inserting the permanent lead 14 through the introducer needle 22 to just beyond the beveled edge 26 of the introducer needle 22. If the curvature of the introducer needle 22 is correct, the permanent lead 14 should pass easily to just beyond the beveled edge 26 of the introducer needle 22. The permanent lead 14 is then removed and the stylet 42 re-inserted into the introducer needle 22.

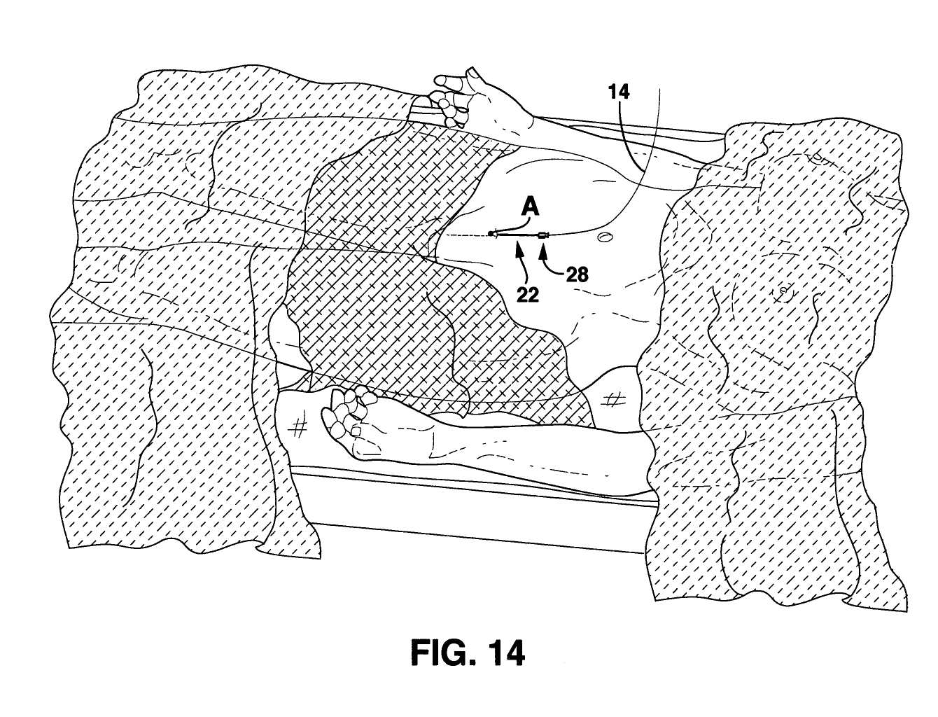

Once the desired position has been reached, the stylet 42 is removed from the introducer needle 22. The permanent lead 14 is slowly inserted through the introducer needle 22 until the distal tip 36 of the permanent lead 14 just exits the introducer needle 22 (FIG. 14). Then, the introducer needle 22 is carefully removed over the permanent lead 14. The permanent lead 14's placement is verified with fluoroscopy. Alternately, the introducer needle 22 can be partially removed. This allows the electrode contacts on the permanent lead 14 to be exposed while facilitating introducer needle 22 reinsertion if repositioning of the permanent lead 14 is needed. Fluoroscopy is used to ensure that all electrodes of the permanent lead 14 are exposed. If necessary, the introducer needle 22 may be adjusted to move the permanent lead 14 to a location where the permanent lead 14 will optimally stimulate the targeted nerve(s).

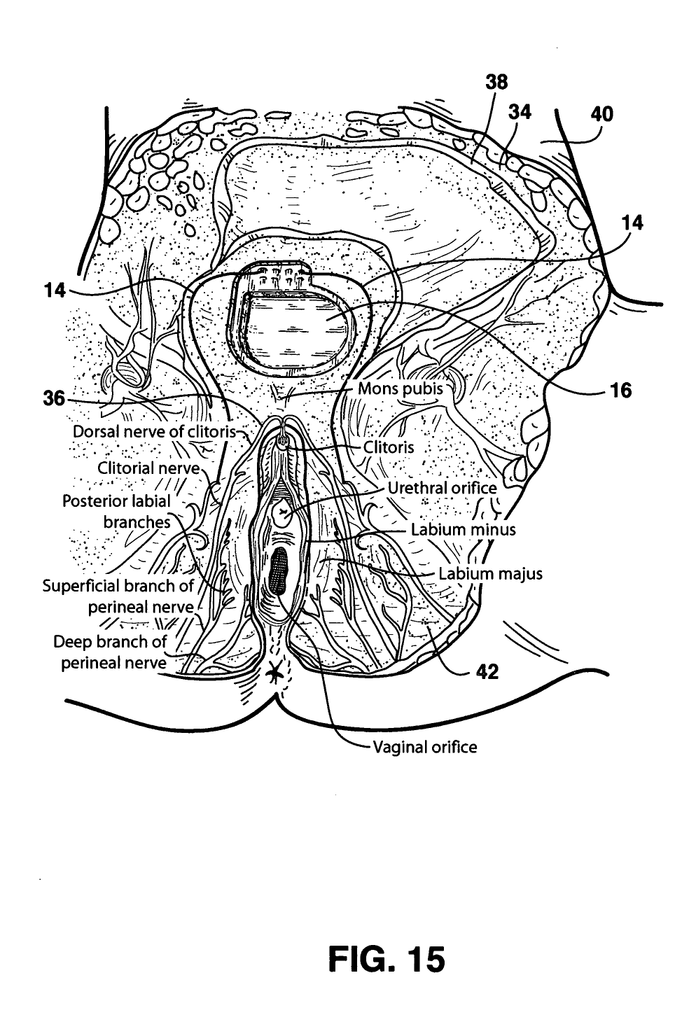

If more than one permanent lead 14 is to be implanted, for example, on each side of the midline to bilateral stimulate desired nerves innervating the vulva, the procedure described above is repeated for each such permanent lead 14 (FIG. 15).

Following placement of the permanent lead 14 by the introducer needle 22, the permanent lead 14 is again connected to the screening device 12, as is well understood in the art. This allows the physician to test the stimulation and confirm that paresthesia is obtained with the placement of the permanent lead 14 prior to permanently implanting the IPG 16 or the RF system receiver 18. Since the patient is preferably awake and alert, the patient will provide verbal feedback regarding paresthesia coverage of the vulva to assess the placement of the permanent lead 14.

If the patient reports muscle contractions (grabbing sensation) or burning, this usually indicates that the electrodes on the permanent lead 14 are too deep in the subcutaneous tissue 34. It may also indicate that the electrodes are significantly above or below the labia majora landmark. It may be necessary to remove and reposition the permanent lead 14. If adjustment of permanent lead 14 is necessary, the screener device 12 is removed from the permanent lead 14. Then, the position of the permanent lead 14 is adjusted and stimulation is tested again.

After good paresthesia coverage is obtained, the screener device 12 is removed from the permanent lead 14. It is now possible to implant the source of electrical stimulation pulses such as the IPG 16 or RF system receiver 18 and any extension sometimes used to connect permanent lead 14 and either IPG 16 or RF system receiver 18 as is well understood in the art. Internalization of the neurostimulation system for nerve stimulation of nerves of the vulva preferably follows the protocol used for other Peripheral Nerve Stimulation (PNS) indications as is well understood in the art. Basically, the procedure involves creating a subcutaneous pocket 24 in tissue (FIG. 15), anchoring the permanent lead 14, implanting the IPG 16 or RF system 18, tunneling the permanent lead 14 and connecting the permanent lead 14 to the IPG 16 or RF system 18 as is well understood in the art.

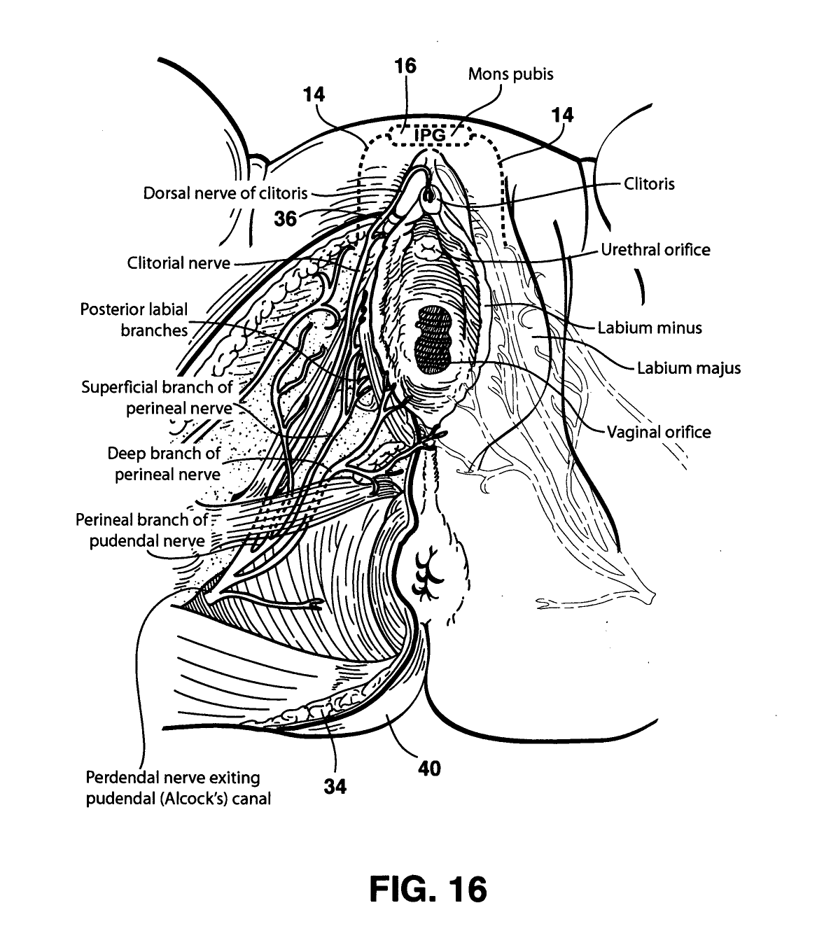

FIG. 16 shows the placement of an alternate embodiment of the IPG 16. This embodiment of IPG 16 is an injectable RF powered implantable stimulator that can be implanted in the subcutaneous tissue 34 via a needle, trocar or surgical opening near a nerve 36 of interest. An example of such an IPG 16 is the Bion® microstimulator sold by Advanced Bionics Corporation of Sylmar, Calif.

The following have been found to be typical ranges for stimulation parameters applied to the permanent lead 14 to obtain optimum paresthesia levels for female sexual dysfunction coverage to treat female sexual dysfunction. These values can vary from patient to patient and may be outside the ranges given here. Never-the-less, these representative values are given for the purpose of illustrating the invention and not for the purpose of limiting the invention. Again, values for these parameters may be higher or lower than the values shown.

| | | |
|---|---|---|
| Amplitude: | 0.5–4.0 | volts |
| Pulse Width: | 90–300 | microseconds |
| Rate: | 50–400 | Hz |

These steps are given as the preferred method of implementing the invention for most patients. It is recognized, however, that the skilled physician will adapt the method described herein using his or her professional skill and judgment to the particular circumstances of a particular patient.

Specific examples of percutaneous nerve stimulation have been given for treating female sexual dysfunction. Although the method of treating female sexual dysfunction has been described in detail, the steps described can be adapted as medical judgment and necessity require.

Further, the method described in detail above has related to treating FSD. Another embodiment of the present invention is to treat intractable pain of the vulva such as vulvodynia, vestibulitis or pain associated with child birth or the trauma of child birth. Women with vulvodynia often have generalized pain of the skin of the vulva and discomfort including itching, stinging, parchedness, dryness, swelling and drawing sensations on the vulvar skin as well as on the skin of and around the rectum.

Vulvodynia also manifests itself as hypersensitivity, pain, itching or stinging particularly in the clitoris, along the edge of the labia minora, in the touching or pulling of pubic hair and in the grooves between the labia majora and labia minora. Vulvodynia pain characteristically is a burning pain that occurs in response to pressure or stretching but can also be residual pain and sometimes constant pain. This pain and discomfort often makes it difficult for the woman to move, walk or even wear underwear. Further, the hypersensitivity of nerves in the vulva can recruit other pain nerves so that pain shoots up the abdomen from the clitoris.

Another type of pain associated with the vulva is vestibulitis. Vestibulitis is an inflammation of the vulvar vestibule (the oval-shaped area that goes from the back of the vaginal opening to just below the clitoris and includes the vaginal and urethral openings), the glands of and around the vagina or connective tissue associated with the vulva. This condition is often characterized by a burning sensation and painful coitus although pain may also be felt on inserting or using tampons or while sitting. Many women with vestibulitis experience a deep, boring or piercing pain in the vestibular glands which also sometimes manifests itself as random stabbing pains.

Vulvar pain also sometimes manifests itself as burning pain along the pubic line, shooting pain through the buttocks and thighs and pain and other parts of the body. In addition, vulvar pain sometimes manifests itself as fibromyalgia, interstitial cystitis or vaginitis with their corresponding symptoms

Further, it is well known that there is pain in the vulvar area associated with labor and trauma caused by childbirth. While labor pain is relatively transient and most women eventually recover from the trauma of childbirth, many women develop chronic pain as a result of pregnancy and the trauma of childbirth.

The present invention contemplates treating vulvar pain whatever its source. This is accomplished by palpating the vulva area to determine the nerve causing the intractable pain, placing a lead superior to that nerve as described above and electrically stimulating the nerve to cause paresthesia of that nerve to ameliorate the pain.

The description contained herein is intended to be illustrative and not exhaustive. Many variations and alternatives of the described technique and method will occur to one of ordinary skill in this art. Further, the hardware described may be varied depending on the physiology and anatomy of a patient as well as the desire of the physician. For example, although single or dual quadripolar as well as single or dual octapolar leads have been described, any type of lead including paddle leads or other types of lead may be used so long as the lead used is able to electrically stimulate the desired nerve. Further, although certain embodiments of an electrical stimulator at been described, it is also within the scope of the invention to use any stimulator, implanted or external, so long as the stimulator is capable of providing a sufficient electrical signal to the lead to stimulate the desired nerves. All these alternatives and variations are intended to be included within the scope of the attached claims. Those familiar with the art may recognize other equivalents to the specific embodiments described herein which equivalents are also intended to be encompassed by the claims attached hereto.

I claim:

1. A method of stimulating peripheral nerves to treat female sexual dysfunction comprising the steps of:
(a) placing a lead having at least one electrode in subcutaneous tissue superior to and near a peripheral nerve that innervates at least a portion of a vulva so that at least one electrode is in the subcutaneous tissue; and
(b) electrically stimulating the peripheral nerve with the at least one electrode in the subcutaneous tissue to cause paresthesia of at least a portion of the vulva.

2. The method of claim 1 wherein the step of placing a lead includes the step of placing a lead across a peripheral nerve that innervates the vulva and that is contributing to female sexual dysfunction.

3. The method of claim 1 wherein the step of placing a lead having at least one electrode in subcutaneous tissue superior to and near a peripheral nerve that innervates at least a portion of a vulva includes the step of placing a lead having at least one electrode in subcutaneous tissue superior to and near a nerve chosen from the group consisting of a pudental nerve, a deep peroneal nerve that innervate the skin, clitoris or medial and inferior aspects of the vulva, an anterior branch of the ilioninguinal nerve innervating the mons pubis or upper part of the labia majora, a genital femoral nerve innervating the labia majora and a posterior femoral cutaneous nerve innervating the more inferoposterior aspects of the vulva.

4. The method of claim 1 wherein the step of placing a lead in the subcutaneous tissue superior to and near a peripheral nerve includes the step of placing a lead in a subcutaneous tissue superior to the peripheral nerve and proximal to a level of detected female sexual dysfunction.

5. The method of claim 1 wherein the step of placing a lead in the subcutaneous tissue superior to and near a peripheral nerve includes the step of subcutaneously placing a lead at the level of the labia majora or vulva across the base of the peripheral nerve trunk and wherein the step of electrically stimulating the peripheral nerve includes the step of electrically stimulating the peripheral nerve trunk.

6. The method of claim 1 further comprising the step of initially palpating the vulva area to identify a specific peripheral nerve that innervates the area of interest in the vulva.

7. The method of claim 1 wherein the step of placing a lead includes the steps of:
(a) providing an introducer needle;
(b) placing the introducer needle in the subcutaneous tissue superior to the peripheral nerve that is innervating at least a portion of the vulva; and
(c) passing, when the introducer needle is in position superior to and near the peripheral nerve, the lead through the introducer needle until the lead is also in position superior to and near the peripheral nerve that is innervating at least a portion of the vulva.

8. The method of claim 7 further comprising the step of removing, after passing the lead through the introducer needle until the lead is also in position superior to and near the peripheral nerve that is innervating at least a portion of the vulva, the introducer needle leaving the lead in place superior to and near the peripheral nerve.

9. The method of claim 8 wherein the step of placing the introducer needle in the subcutaneous tissue superior to the peripheral nerve that is innervating at least a portion of the vulva includes the step of curving the introducer needle to conform to the contour of the patient's body superior to the peripheral nerve.

10. The method of claim 1 wherein the step of placing a lead includes the step of placing dual leads.

11. The method of claim 10 wherein the step of placing dual leads includes the step of placing a lead on either side of a clitoris.

12. The method of claim 11 wherein the step of placing a lead on either side of a clitoris includes the step of placing a lead within the labia majora.

13. The method of claim 1 wherein the step of electrically stimulating the peripheral nerve further includes the steps of
(a) connecting the lead to a device for producing an electrical signal; and
(b) producing an electrical signal to produce electrical stimulation.

14. The method of claim 13 wherein the device for producing an electrical signal is chosen from the group consisting of a an implanted pulse generator or an implanted RF system receiver and its corresponding RF system transmitter.

15. The method of claim 13 wherein the step of electrically stimulating the peripheral nerve lead includes the step of electrically stimulating the patient with an electrical signal having an amplitude between about 0.5 to about 4.0 volts.

16. The method of claim 13 wherein the step of electrically stimulating the peripheral nerve includes the step of electrically stimulating the patient with an electrical signal having a rate between about 50 Hz. to about 400 Hz.

17. The method of claim 13 wherein the step of electrically stimulating the peripheral nerve includes the step of electrically stimulating the patient with an electrical signal having a pulse width between about 90 microseconds to about 300 microseconds.

18. The method of claim 1 wherein the step of electrically stimulating the peripheral nerve further includes the step of connecting the lead to a screening device.

19. The method of claim 18 wherein the step of electrically stimulating the peripheral nerve further comprising the step of performing test electrical stimulation with the lead.

20. The method of claim 19 wherein the step of performing test electrical stimulation with the lead includes the steps of:
(a) electrically stimulating the patient by the lead and screener device;
(b) getting a patient's feedback to the step of electrically stimulating the patient; and
(c) evaluating the lead position from the patient's feedback to the step of electrically stimulating the patient.

21. The method of claim 20 wherein the step of getting a patient's feedback to the step of electrically stimulating the patient includes the step of receiving verbal feedback from the patient regarding paresthesia coverage of the effects of the electrical stimulation by the lead.

22. The method of claim 19′ wherein the step of performing test electrical stimulation with the lead includes the steps of:
(a) electrically stimulating the patient by the lead and screener device;
(b) getting a patient's feedback to the step of electrically stimulating the patient; and
(c) developing optimal stimulation parameters based on the patient's response to the step of electrically stimulating the patient.

23. The method of claim 22 wherein the step of getting a patient's feedback to the step of electrically stimulating the patient includes the step of receiving verbal feedback from the patient regarding paresthesia coverage of the effects of the electrical stimulation by the lead.

24. The method of claim 19 wherein the step of performing test electrical stimulation with the lead includes the step of electrically stimulating the patient with an electrical signal having an amplitude between about 0.5 to about 4.0 volts.

25. The method of claim 19 wherein the step of performing test electrical stimulation with the lead includes the step of electrically stimulating the patient with an electrical signal having a rate between about 50 Hz. to about 400 Hz.

26. The method of claim 19 wherein the step of performing test electrical stimulation with the lead includes the step of electrically stimulating the patient with an electrical signal having a pulse width between about 90 microseconds to about 300 microseconds.

27. The method of claim 1 wherein the step of electrically stimulating the peripheral nerve includes the steps of:
(a) implanting an implantable pulse generator;
(b) electrically connecting the implantable pulse generator to the lead; and
(c) producing an electrical signal to produce electrical stimulation.

28. The method of claim 1 wherein the step of electrically stimulating the peripheral nerve includes the steps of:
(a) implanting a RF system receiver;
(b) electrically connecting the RF system receiver to the lead; and
(c) producing an electrical signal to produce electrical stimulation.

29. The method of claim 1 wherein:
(a) the step of placing a lead near a peripheral nerve that is innervating at least a portion of the vulva includes the step of placing a screening lead near a peripheral nerve that is innervating at least a portion of the vulva; and
(b) the step of electrically stimulating the peripheral nerve with the lead to cause paresthesia of at least a portion of the vulva area includes the step of electrically stimulating the peripheral nerve with the screening lead; and
further comprising the steps of
(c) placing a permanent lead near a peripheral nerve that is innervating at least a portion of the vulva; and
(d) electrically stimulating the peripheral nerve with the permanent lead to cause paresthesia of at least a portion of the vulva.

30. The method of claim 29 wherein the step of placing a screening lead includes the step of placing a screening lead across a peripheral nerve that innervates the vulva and that is contributing to female sexual dysfunction.

31. The method of claim 29 wherein the step of placing a permanent lead includes the step of placing a permanent lead across a peripheral nerve that innervates the vulva and that is contributing to female sexual dysfunction.

32. The method of claim 29 wherein the step of placing a lead having at least one electrode in subcutaneous tissue superior to and near a peripheral nerve that innervates at least a portion of a vulva includes the step of placing a lead having at least one electrode in subcutaneous tissue superior to and near a nerve chosen from the group consisting of a pudental nerve, a deep peroneal nerve that innervate the skin, clitoris or medial and inferior aspects of the vulva, an anterior branch of the ilioninguinal nerve innervating the mons pubis or upper part of the labia majora, a genital femoral nerve innervating the labia majora and a posterior femoral cutaneous nerve innervating the more inferoposterior aspects of the vulva.

33. The method of claim 29 wherein the step of placing a lead in the subcutaneous tissue superior to and near a peripheral nerve includes the step of placing a lead in a subcutaneous tissue superior to the peripheral nerve and proximal to a level of detected female sexual dysfunction.

34. The method of claim 29 wherein the step of placing a lead in the subcutaneous tissue superior to and near a peripheral nerve includes the step of subcutaneously placing a lead at the level of the labia majora or vulva across the base of the peripheral nerve trunk and wherein the step of electrically stimulating the peripheral nerve includes the step of electrically stimulating the peripheral nerve trunk.

35. The method of claim 29 further comprising the step of initially palpating the vulva area to identify a specific peripheral nerve that innervates the area of interest in the vulva.

36. The method of claim 29 wherein the step of placing a lead includes the steps of:
(a) providing an introducer needle;
(b) placing the introducer needle in the subcutaneous tissue superior to the peripheral nerve that is innervating at least a portion of the vulva; and
(c) passing, when the introducer needle is in position superior to and near the peripheral nerve, the lead through the introducer needle until the lead is also in position superior to and near the peripheral nerve that is innervating at least a portion of the vulva.

37. The method of claim 36 further comprising the step of removing, after passing the lead through the introducer needle until the lead is also in position superior to and near the peripheral nerve that is innervating at least a portion of the vulva, the introducer needle leaving the lead in place superior to and near the peripheral nerve.

38. The method of claim 37 wherein the step of placing the introducer needle in the subcutaneous tissue superior to the peripheral nerve that is innervating at least a portion of the vulva includes the step of curving the introducer needle to conform to the contour of the patient's body superior to the peripheral nerve.

39. The method of claim 29 wherein the step of placing a lead includes the step of placing dual leads.

40. The method of claim 39 wherein the step of placing dual leads includes the step of placing a leads on either side of a clitoris.

41. The method of claim 40 wherein the step of placing a lead on either side of a clitoris includes the step of placing a lead within the labia majora.

42. The method of claim 29 wherein the step of electrically stimulating the peripheral nerve further includes the steps of
(a) connecting the lead to a device for producing an electrical signal; and
(b) producing an electrical signal to produce electrical stimulation.

43. The method of claim 42 wherein the device for producing an electrical signal is chosen from the group consisting of a an implanted pulse generator or an implanted RF system receiver and its corresponding RF system transmitter.

44. The method of claim 42 wherein the step of electrically stimulating the peripheral nerve lead includes the step of electrically stimulating the patient with an electrical signal having an amplitude between about 0.5 to about 4.0 volts.

45. The method of claim 42 wherein the step of electrically stimulating the peripheral nerve includes the step of electrically stimulating the patient with an electrical signal having a rate between about 50 Hz. to about 400 Hz.

46. The method of claim 42 wherein the step of electrically stimulating the peripheral nerve includes the step of electrically stimulating the patient with an electrical signal having a pulse width between about 90 microseconds to about 300 microseconds.

47. The method of claim 29 wherein the step of electrically stimulating the peripheral nerve further includes the step of connecting the lead to a screening device.

48. The method of claim 47 wherein the step of electrically stimulating the peripheral nerve further comprising the step of performing test electrical stimulation with the lead.

49. The method of claim 48 wherein the step of performing test electrical stimulation with the lead includes the steps of:
(a) electrically stimulating the patient by the lead and screener device;
(b) getting a patient's feedback to the step of electrically stimulating the patient; and
(c) evaluating the lead position from the patient's feedback to the step of electrically stimulating the patient.

50. The method of claim 49 wherein the step of getting a patient's feedback to the step of electrically stimulating the patient includes the step of receiving verbal feedback from the patient regarding paresthesia coverage of the effects of the electrical stimulation by the lead.

51. The method of claim 48 wherein the step of performing test electrical stimulation with the lead includes the steps of:
(a) electrically stimulating the patient by the lead and screener device;
(b) getting a patient's feedback to the step of electrically stimulating the patient; and
(c) developing optimal stimulation parameters based on the patient's response to the step of electrically stimulating the patient.

52. The method of claim 51 wherein the step of getting a patient's feedback to the step of electrically stimulating the patient includes the step of receiving verbal feedback from the patient regarding paresthesia coverage of the effects of the electrical stimulation by the lead.

53. The method of claim 48 wherein the step of performing test electrical stimulation with the lead includes the step of electrically stimulating the patient with an electrical signal having an amplitude between about 0.5 to about 4.0 volts.

54. The method of claim 48 wherein the step of performing test electrical stimulation with the lead includes the step of electrically stimulating the patient with an electrical signal having a rate between about 50 Hz. to about 400 Hz.

55. The method of claim 48 wherein the step of performing test electrical stimulation with the lead includes the step of electrically stimulating the patient with an electrical signal having a pulse width between about 90 microseconds to about 300 microseconds.

56. The method of claim 29 wherein the step of electrically stimulating the peripheral nerve includes the steps of:
(a) implanting an implantable pulse generator;
(b) electrically connecting the implantable pulse generator to the lead; and
(c) producing an electrical signal to produce electrical stimulation.

57. The method of claim 29 wherein the step of electrically stimulating the peripheral nerve includes the steps of:
(a) implanting a RF system receiver;
(b) electrically connecting the RF system receiver to the lead; and
(c) producing an electrical signal to produce electrical stimulation.

58. A method of stimulating peripheral nerves to treat female sexual dysfunction comprising the steps of:
(a) placing a lead having at least one electrode in subcutaneous tissue superior to and near a peripheral nerve that innervates at least a portion of a vulva so that at least one electrode is in the subcutaneous tissue wherein the step of placing a lead includes the steps of:
(i) providing an introducer needle;
(ii) placing the introducer needle in the subcutaneous tissue superior to the peripheral nerve that is innervating at least a portion of the vulva; and
(iii) passing, when the introducer needle is in position superior to and near the peripheral nerve, the lead through the introducer needle until the lead is also in position superior to and near the peripheral nerve that is innervating at least a portion of the vulva;
(iv) placing a lead through the introducer needle across a peripheral nerve that innervates the vulva and that is contributing to female sexual dysfunction, the lead having at least one electrode placed in subcutaneous tissue superior to and near a nerve chosen from the group consisting of a pudental nerve, a deep peroneal nerve that innervate the skin; clitoris or medial and inferior aspects of the vulva, an anterior branch of the ilioninguinal nerve innervating the mons pubis or upper part of the labia majora, a genital femoral nerve innervating the labia majora and a posterior femoral cutaneous nerve innervating the more inferoposterior aspects of the vulva; and
(b) electrically stimulating the peripheral nerve with the at least one electrode in the subcutaneous tissue to cause paresthesia of at least a portion of the vulva.

59. The method of claim 58 further comprising the step of initially palpating the vulva area to identify a specific peripheral nerve that innervates the area of interest in the vulva.

60. A method of stimulating a peripheral nerve to treat female sexual dysfunction emanating from the peripheral nerve, the peripheral nerve chosen from a group consisting of a pudental nerve, a deep peroneal nerve, a clitoral nerve, the anterior branch of the ilioninguinal nerve innervating the mons pubis and upper part of the labia majora, the genital femoral nerve innervating the labia majora and the posterior femoral cutaneous nerve supplying the more inferoposterior aspects of the vulva, the method comprising the steps of:
(a) placing a lead having at least one electrode in subcutaneous tissue superior to and near a peripheral nerve that innervates at least a portion of a vulva so that at least one electrode is in the subcutaneous tissue; and
(b) electrically stimulating the peripheral nerve with the at least one electrode in the subcutaneous tissue to cause paresthesia of at least a portion of the vulva.

61. A method of stimulating peripheral nerves to treat female sexual dysfunction comprising the steps of:
(a) providing an introducer needle;
(b) providing a lead having at least one electrode;
(c) placing the introducer needle in the subcutaneous tissue superior to and near a peripheral nerve that is innervating at least a portion of the vulva and that is contributing to female sexual dysfunction;
(d) passing, when the introducer needle is in position superior to and near the peripheral nerve that is innervating at least a portion of the vulva, the lead through the introducer needle so that at least one electrode of the lead is also in subcutaneous tissue in position superior to and near the peripheral nerve that is innervating at least a portion of the vulva;
(e) electrically stimulating the peripheral nerve with the at least one electrode in the subcutaneous tissue to cause paresthesia of at least a portion of the vulva.

62. The method of claim 61 wherein:
(a) the step of providing a lead having at least one electrode includes the step of providing a screening lead having at least one electrode and the step of providing a permanent lead having at least one electrode;
(b) the step of passing the lead through the introducer needle includes the step of passing the screening lead through the introducer needle so that at least one electrode of the screening lead is also in subcutaneous tissue in position superior to and near a peripheral nerve that is innervating at least a portion of the vulva;
(c) the step of electrically stimulating the peripheral nerve with the lead to cause paresthesia of at least a portion of the vulva area includes the step of electrically stimulating the peripheral nerve with the screening lead;
(d) the step of passing the lead through the introducer needle includes the step of removing the screening lead through the introducer needle and passing the permanent lead through the introducer needle so that at least one electrode of the permanent lead is also in subcutaneous tissue in position superior to and near a peripheral nerve that is innervating at least a portion of the vulva;
(e) the step of electrically stimulating the peripheral nerve with the lead to cause paresthesia of at least a portion of the vulva area includes the step of electrically stimulating the peripheral nerve with the permanent lead.

63. The method of claim 61 wherein the step of placing the introducer needle in the subcutaneous tissue superior to and near a peripheral nerve that is innervating at least a portion of the vulva and that is contributing to female sexual dysfunction includes the step of placing the introducer needle in tissue superior to and near a peripheral nerve that is innervating at least a portion of the vulva and that is contributing to female sexual dysfunction chosen from the group consisting of a pudental nerve, a deep peroneal nerve that innervate the skin, clitoris or medial and inferior aspects of the vulva, an anterior branch of the ilioninguinal nerve innervating the mons pubis or upper part of the labia majora, a genital femoral nerve innervating the labia majora and a posterior femoral cutaneous nerve innervating the more inferoposterior aspects of the vulva.

64. A method of treating neuralgias of or associated with a vulva comprising the steps of:
(a) placing a lead having at least one electrode in subcutaneous tissue superior to and near the peripheral nerve innervating at least a portion of the vulva and that is causing the neuralgia emanating from the peripheral nerve so that at least one electrode is in the subcutaneous tissue; and
(b) electrically stimulating the peripheral nerve with the at least one electrode in the subcutaneous to cause paresthesia of the area producing the neuralgia.

65. A method of stimulating peripheral nerves to treat neuralgias emanating from a peripheral nerve, the neuralgias chosen from a group consisting of post herpetic neuralgia, chronic deafferentation female sexual dysfunction, chronic peripheral nerve female sexual dysfunction, post craniotomy female sexual dysfunction, incisional female sexual dysfunction, clunial nerve female sexual dysfunction, post hemiorrhapy female sexual dysfunction, localized low back or other spine female sexual dysfunction, incisional neuroma female sexual dysfunction, stump neuroma female sexual dysfunction, incisional scar female sexual dysfunction, deafferention female sexual dysfunction, chronic peripheral nerve female sexual dysfunction, sciatic neuralgia, medial neuralgia and ulnar neuralgia comprising the steps of:
(a) placing a lead having at least one electrode in subcutaneous tissue superior to and near a peripheral nerve that innervates at least a portion of a vulva so that at least one electrode is in the subcutaneous tissue; and
(b) electrically stimulating the peripheral nerve with the at least one electrode in the subcutaneous tissue to cause paresthesia of at least a portion of the vulva.

66. A method of treating neuralgias of or associated with a vulva comprising the steps of:
(a) placing a lead having at least one electrode in subcutaneous tissue superior to and near the peripheral nerve innervating at least a portion of the vulva and that is causing the neuralgia emanating from the peripheral nerve so that at least one electrode is in the subcutaneous tissue, the peripheral nerve chosen from the group consisting of branches of the pudental and deep peroneal nerves that innervate the skin, clitoris and medial and inferior aspects of the vulva, the anterior branch of the ilioninguinal nerve innervating the mons pubis and upper part of the labia majora, the genital femoral nerve innervating the labia majora and the posterior femoral cutaneous nerve supplying the more inferoposterior aspects of the vulva; and
(b) electrically stimulating the peripheral nerve with the at least one electrode in the subcutaneous to cause paresthesia of the area producing the neuralgia.
